(19) United States
(12) Patent Application Publication
(10) Pub. No.: US 2009/0186053 A1
(43) Pub. Date: Jul. 23, 2009

(54) Surface-Modified Non-Metal/Metal Oxides Coated With Silicon Dioxide

(75) Inventors: Jürgen Meyer, Stockstadt (DE); Steffen Hasenzahl, Hanau (DE); Kai Schumacher, Hofheim (DE)

Correspondence Address: SMITH, GAMBRELL & RUSSELL, SUITE 3100, PROMENADE II, 1230 PEACHTREE STREET, N.E., ATLANTA, GA, 30309-3592, US

(73) Assignee: DEGUSSA AG, Dusseldorf (DE)

(21) Appl. No.: 10/597,419

(22) PCT Filed: Jan. 21, 2005
(86) PCT No.: PCT/EP2005/000576

§ 371 Date: Jun. 26, 2008

(30) Foreign Application Priority Data
Jan. 28, 2004 (DE) 10 2004 004 147.4

(51) Int. Cl. A61K 8/02; B32B 15/02; A61Q 17/04

(52) U.S. Cl. 424/401; 428/404; 424/59

(57) ABSTRACT

Surface-modified metal oxide particles coated with silicon dioxide and having a low structure are produced by adding a base dissolved in water, with stirring, to a dispersion consisting of a metal oxide, at least one compound of the type XₙSi(OR)₄₋ₙ and water, separating off, optionally washing with water, drying and surface-modifying the reaction product. The surface-modified metal oxide particles coated with silicon dioxide can be used in sunscreens and in CMP applications.

The invention concerns surface-modified non-metal/metal oxides coated with silicon dioxide, a process for their production, and their use.

Metal oxides, such as titanium dioxide or zinc oxide, are widely used in sunscreens. Their action is substantially based on reflection, scattering and absorption of damaging UV radiation and substantially depends on the primary particle size of the metal oxides.

Metal oxides such as titanium dioxide or zinc oxide display photocatalytic activity.

A known means of reducing the photocatalytic activity is to produce metal oxide particles coated with silicon dioxide for use as a component in sunscreens.

The disadvantage, however, is that these coated metal oxide particles display a low surface functionality and a high degree of intergrowth of the particles, making it more difficult to incorporate the particles into a cosmetic formulation and also limiting their stability with regard to sedimentation. A further disadvantage is that in addition to water an organic solvent is absolutely essential in the production of these particles in order for a shell to form. Along with increased safety precautions, this solvent also requires additional expense in order to separate it from the water again after the reaction and/or to dispose of it.

The object of the invention is to provide coated non-metal/metal oxide particles which do not display the disadvantages of the prior art, can be readily incorporated into cosmetic formulations, are stable in these and display a low photocatalytic activity.

Another object is to provide a process for the production of coated non-metal/metal oxide particles which does not display the disadvantages of the prior art.

The invention provides surface-modified coated oxide particles, consisting of a core of a non-metal/metal oxide and a shell of silicon dioxide surrounding the core, wherein the coated oxide particles display a low structure, defined by the absence of an end point in dibutyl phthalate absorption.

The surface modification can be performed by spraying the coated non-metal/metal oxides with the surface-modifying agent at room temperature and then heat treating the mixture at a temperature of 50 to 400° C. for a period of 1 to 6 hours.

An alternative method of surface modification of the coated non-metal/metal oxides can be performed by treating the coated non-metal/metal oxides with the surface-modifying agent in vapour form and then heat treating the mixture at a temperature of 50 to 800° C. for a period of 0.5 to 6 hours.

The heat treatment can take place under protective gas, such as nitrogen for example.

The surface modification can be performed in heatable mixers and dryers with sprayers, continuously or in batches. Suitable devices can be, for example: ploughshare mixers, plate dryers, fluidised-bed or flash dryers.

The surface modification can be performed with known agents such as are used for the surface modification and/or silanisation of oxides.

The following substances or mixtures of substances can be used:
a) Organosilanes of the type (RO)₃Si(CₙH₂ₙ₊₁) and (RO)₃Si(CₙH₂ₙ₋₁)
   R=alkyl, such as e.g. methyl, ethyl, n-propyl, i-propyl, butyl
   n=1-20
b) Organosilanes of the type R′ₓ(RO)_{y}Si(CₙH₂ₙ₊₁) and Rₓ′ (RO)_{y}Si(CₙH₂ₙ₋₁)
   R=alkyl, such as e.g. methyl, ethyl, n-propyl, i-propyl, butyl
   R′=alkyl, such as e.g. methyl, ethyl, n-propyl, i-propyl, butyl
   R′=cycloalkyl
   n=1-20
   x+y=3
   x=1, 2
   y=1, 2
c) Organohalosilanes of the type X₃Si(CₙH₂ₙ₊₁) and X₃Si(CₙH₂ₙ₋₁)
   X=Cl, Br
   n=1-20
d) Organohalosilanes of the type X₂(R′)Si(CₙH₂ₙ₊₁) and X₂(R′)Si(CₙH₂ₙ₋₁)
   X=Cl, Br
   R′=alkyl, such as e.g. methyl, ethyl, n-propyl, i-propyl, butyl
   R′=cycloalkyl
   n=1-20
e) Organohalosilanes of the type X(R′)₂Si(CₙH₂ₙ₊₁) and X(R′)₂Si(CₙH₂ₙ₋₁)
   X=Cl, Br
   R′=alkyl, such as e.g. methyl, ethyl, n-propyl, i-propyl, butyl
   R′=cycloalkyl
   n=1-20
f) Organosilanes of the type (RO)₃Si(CH₂)ₘ—R′
   R=alkyl, such as methyl, ethyl, propyl
   m=0, 1-20
   R′=methyl, aryl (e.g. —C₆H₅, substituted phenyl radicals)
      —C₄F₉, OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃
      —SH
      —NR′R″R′″(R′=alkyl, aryl; R″=H, alkyl, aryl; R′″=H, alkyl, aryl, benzyl, C₂H₄NR″″R′″″ where R″″=A, alkyl and R′″″=H, alkyl)
g) Organosilanes of the type (R″)ₓ(RO)_{y}Si(CH₂)ₘ—R′

$$\begin{matrix}{R}^{″} = alkyl \\ = cycloakyl\end{matrix}$$

$$x + y = 3$$

$$x = 1 , 2$$

$$y = 1 , 2$$

$$m = 0 , 1 to 20$$

R′=methyl, aryl (e.g. —C₆H₅, substituted phenyl radicals)
   —C₄F₉, —OCF₂—CHF—CF₃, —C₆F₁₃, —O—CF₂—CHF₂
   —NH₂, —N₃, —SCN, —CH═CH₂, —NH—CH₂—CH₂—NH₂,
   —N—(CH₂—CH₂—NH₂)₂
   —OOC(CH₃)C═CH₂
   —OCH₂—CH(O)CH₂
   —NH—CO—N—CO—(CH₂)₅
   —NH—COO—CH₃, —NH—COO—CH₂—CH₃, —NH—(CH₂)₃Si(OR)₃
   —Sₓ—(CH₂)₃Si(OR)₃
   —SH—NR′R″R′″(R′=alkyl, aryl; R″=H, alkyl, aryl; R′″=H, alkyl, aryl, benzyl, C₂H₄NR″″R′″″ where R″″=A, alkyl and R′″″=H, alkyl)
h) Organohalosilanes of the type X₃Si(CH₂)ₘ—R′
   X=Cl, Br
   m=0, 1-20
   R′=methyl, aryl (e.g. —C₆H₅, substituted phenyl radicals)
      —C₄F₉, —OCF₂—CHF—CF₃, —C₆F₁₃, —O—CF₂—CHF₂
      —NH₂, —N₃, —SCN, —CH═CH₂,
      —NH—CH₂—CH₂—NH₂
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃
      —SH
i) Organohalosilanes of the type (R)X₂Si(CH₂)ₘ—R′
   X=Cl, Br
   R=alkyl, such as methyl, ethyl, propyl
   m=0, 1-20
   R′=methyl, aryl (e.g. —C₆H₅, substituted phenyl radicals)
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃, where R can be methyl, ethyl, propyl, butyl
      —Sₓ—(CH₂)₃Si(OR)₃, where R can be methyl, ethyl, propyl, butyl
      —SH
j) Organohalosilanes of the type (R)₂XSi(CH₂)ₘ—R′
   X=Cl, Br
   R=alkyl
   m=0, 1-20
   R′=methyl, aryl (e.g. —C₆H₅, substituted phenyl radicals)
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃
      —SH
k) Silazanes of the type

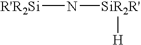

R=alkyl
R′=alkyl, vinyl
l) Cyclic polysiloxanes of the type D 3, D 4, D 5, wherein D 3, D 4 and D 5 are understood to be cyclic polysiloxanes having 3, 4 or 5 units of the type —O—Si (CH₃)₂—,
   e.g. octamethyl cyclotetrasiloxane=D 4

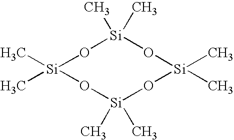

m) Polysiloxanes or silicone oils of the type

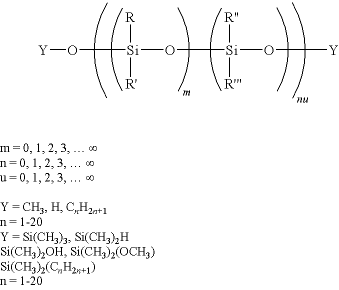

R=alkyl, such as CₙH₂ₙ₊₁, wherein n=1 to 20, aryl, such as phenyl and substituted phenyl radicals, (CH₂)ₙ—NH₂, H
R′=alkyl, such as CₙH₂ₙ₊₁, wherein n=1 to 20, aryl, such as phenyl and substituted phenyl radicals, (CH₂)ₙ—NH₂, H
R″=alkyl, such as CₙH₂ₙ₊₁, wherein n=1 to 20, aryl, such as phenyl and substituted phenyl radicals, (CH₂)ₙ—NH₂, H
R′″=alkyl, such as CₙH₂ₙ₊₁, wherein n=1 to 20, aryl, such as phenyl and substituted phenyl radicals, (CH₂)ₙ—NH₂, H

The following substances can preferably used as surface-modifying agents:

Octyl trimethoxysilane, octyl triethoxysilane, hexamethyl disilazane, 3-methacryloxypropyl trimethoxysilane, 3-methacryloxypropyl triethoxysilane, hexadecyl trimethoxysilane, hexadecyl triethoxysilane, dimethyl polysiloxane, glycidyloxypropyl trimethoxysilane, glycidyloxypropyl triethoxysilane, nonafluorohexyl trimethoxysilane, tridecafluorooctyl trimethoxysilane, tridecafluorooctyl triethoxysilane, aminopropyl triethoxysilane.

Octyl trimethoxysilane, octyl triethoxysilane and dimethyl polysiloxanes can particularly preferably be used.

The term structure can be understood to be the degree of intergrowth of the particles, which can be measured by DBP absorption (dibutyl phthalate absorption).

Low structure is manifested by the fact that no end point can be detected in DBP absorption. This denotes a low degree of intergrowth of the particles.

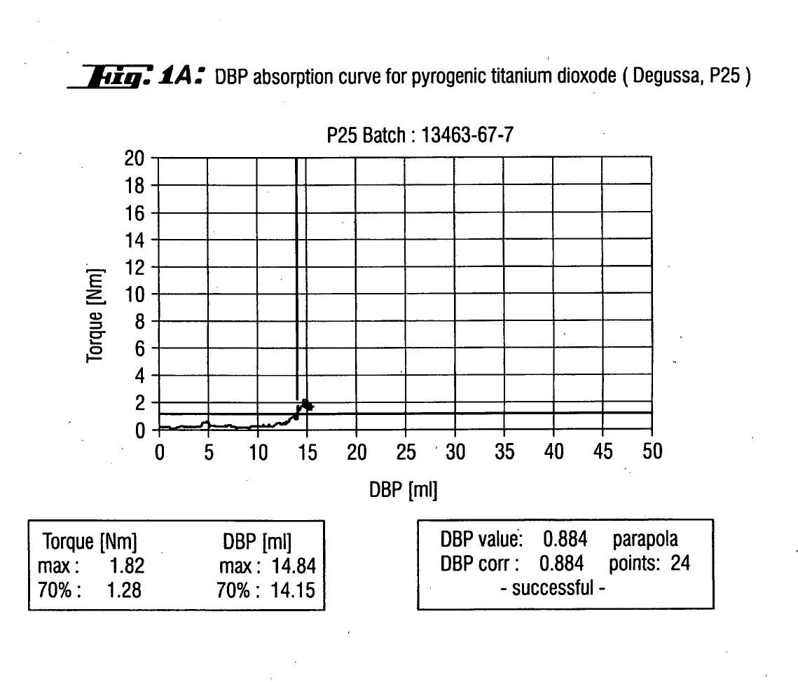

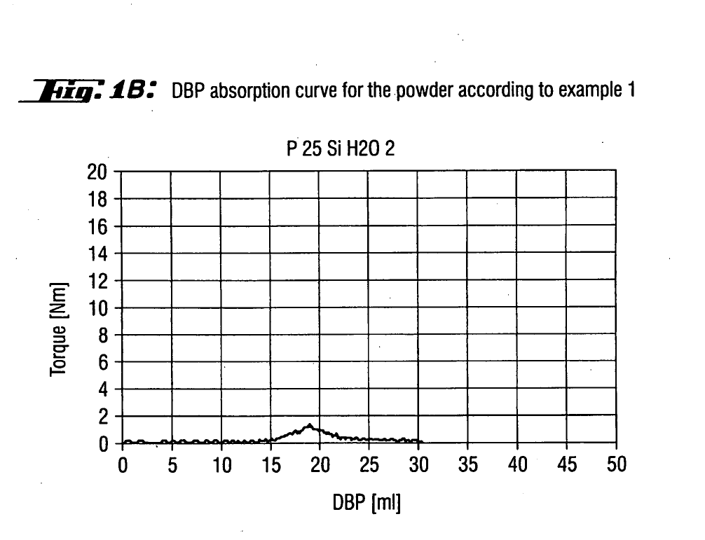

In DBP absorption the force take-up, or the torque (in Nm), of the rotating blades of the DBP measuring device is measured during the addition of defined amounts of dibutyl phthalate. For non-metal/metal oxides (e.g. titanium dioxide or silicon dioxide, FIG. 1A) the addition of a specific amount of dibutyl phthalate produces a sharply defined maximum with a subsequent drop. In the case of the particles used according to the invention, a maximum with a subsequent drop is not detected, which means that the device cannot establish an end point (FIG. 1B).

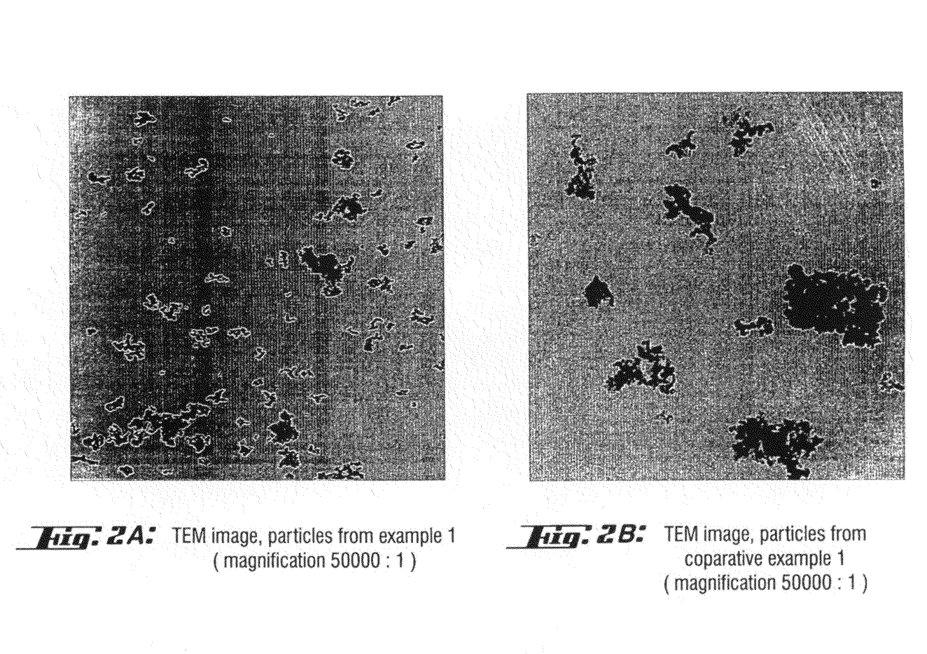

The low structure of the particles used according to the invention can also be seen from the TEM images (FIG. 2A). The known particles, produced according to EP-A-0 988 853, display markedly greater aggregation (FIG. 2B).

The particles used according to the invention preferably display a photocatalytic activity of less than K=0.20·10⁻³ mol kg⁻¹·min⁻¹.

The activity is determined by the oxidation of 2-propanol to acetone by irradiation with UV light. The result is expressed as the rate of formation of acetone in the form of a speed constant, zero order K=dc(Ac)dt.

The measurement is based on the method disclosed by Robert Rudham in “The Chemistry of Physical Sunscreen Materials” (Review derived from a presentation made at the FDA Workshop on the Photochemistry and Photobiology of Sunscreens, Washington, Sep. 19-20, 1996). The low photocatalytic activity means that the oxide particles used according to the invention can be used in sunscreens.

The BET surface area, determined in accordance with DIN 66131, of the particles used according to the invention can be varied in a broad range between 5 and 600 m²/g. The BET surface area of the particles used according to the invention is usually greater than that of the underlying core material. With different production conditions, however, it can optionally also be less than that of the core material used. The BET surface area of the particles used according to the invention is preferably greater than that of the underlying cores, however.

The primary particle size of the coated oxide particles can be between 2 and 100 nm, preferably between 5 and 50 nm, and the secondary particle size can be between 0.05 and 50 μm, preferably between 0.1 and 1 μm. In these ranges, when used in sunscreens, the particles used according to the invention display an adequate UV protection and a pleasant feel on the skin after application.

These particle sizes are determined in accordance with DIN 53206.

The film thickness of the silicon dioxide shell of the metal oxide particles used according to the invention can be varied between 0.5 and 25 nm.

The non-metal/metal oxide particles can be titanium dioxide, zinc oxide, zirconium oxide, iron oxide, cerium oxide and/or chemical mixtures (mixed oxides) of these metal oxides with one another, and/or chemical mixtures (mixed oxides) of these metal oxides with aluminium oxide and/or chemical mixtures (mixed oxides) of these metal oxides with silicon dioxide. They can be non-metal/metal oxides derived from a pyrogenic process, preferably flame hydrolysis, a sol gel, a plasma process, a precipitation process, a hydrothermal process or combinations of the above processes.

Particularly preferred metal oxides are the pyrogenically produced metal oxides titanium dioxide, zinc oxide, iron oxide, cerium oxide, zirconium oxide and/or chemical mixtures (mixed oxides) of these metal oxides with one another, and/or chemical mixtures (mixed oxides) of these metal oxides with aluminium oxide and/or chemical mixtures (mixed oxides) of these metal oxides with silicon dioxide.

Chemical mixtures of pyrogenically produced oxides should be understood to be, for example, those in which a component is incorporated into the pyrogenic process via an aerosol, as described in EP-B-0 850 876. Both components can also be evaporated simultaneously and introduced into the mixing chamber of a burner, such as is used to manufacture pyrogenically produced oxides. This is described for example in EP-A-609 533 for titanium-silicon mixed oxide and titanium-aluminium mixed oxide or in EP-A-1 048 617 for silicon-aluminium mixed oxide.

A pyrogenically produced metal oxide can also be coated or partially coated with another metal oxide, which is applied to the pyrogenically produced metal oxide in a non-pyrogenic process.

In a process for producing the oxide particles used according to the invention, a base dissolved in water is added with stirring to a dispersion consisting of 1-80 wt. % of a metal oxide, at least one compound of the type XₙSi(OR)₄₋ₙ, wherein the molar ratio XₙSi(OR)₄₋ₙ/metal oxide is between 0.1 and 25, depending on the film thickness of the silicon dioxide shell, and water, the reaction product is separated off, optionally washed and dried.

As compounds of the type XₙSi(OR)₄₋ₙ those in which X=halogen or H, R=H or a linear or a branched alkyl radical having 1 to 8 C atoms and n=0-4, where R does not equal H if n=4, are preferably used. Tetraalkoxysilanes and/or oligomers thereof are particularly preferred.

The reaction product can be separated off by filtration or centrifuging. It can be washed with water, an organic solvent, or mixtures of water with organic solvents, water being preferred within the meaning of the invention.

The particles used according to the invention can be dried by methods known to the person skilled in the art. An overview of various drying methods can be found in Ullmann's Encyclopedia of Industrial Chemistry, Vol. B2, Unit Operations 1, pages 4-2 to 4-35, 5^{th} edition.

This can be followed by further process steps, such as e.g. calcination, grinding processes, granulation processes, or dispersion in suitable liquid media.

The temperature at which the reaction is performed is not critical, provided that the reaction medium is liquid. A reaction temperature of 15 to 30° C. is preferred.

The amount of base that is required can be varied across a wide range, from 0.1 to 30 wt. %, relative to the overall reaction medium. A base concentration of 1 to 5 wt. % can be particularly advantageous, since at a low base concentration there is a rapid formation of the oxide particles according to the invention.

Bases that can be used are ammonia; hydroxides, such as sodium hydroxide, potassium hydroxide or tetraalkyl ammonium hydroxide; carbonates, such as ammonium carbonate, ammonium hydrogen carbonate, sodium carbonate or sodium hydrogen carbonate; organic bases, such as amines, pyridines, anilines, guanidine; ammonium salts of carboxylic acids, such as ammonium formate, ammonium acetate; alkyl ammonium salts of carboxylic acids, such as monomethylamine formate, dimethylamine formate and mixtures thereof.

Particularly preferred are ammonia, ammonium carbonate, ammonium hydrogen carbonate, ammonium formate, ammonium acetate, sodium carbonate and sodium hydrogen carbonate and mixtures of two or more of these compounds.

In addition to bases, inorganic acids, such as e.g. hydrochloric acid, sulfuric acid or phosphoric acid, and organic acids, such as formic or acetic acid, can also be used, in order to release silicon dioxide from the silicon dioxide source.

The non-metal/metal oxide particles can be titanium dioxide, zinc oxide, zirconium oxide, iron oxide, cerium oxide and/or chemical mixtures (mixed oxides) of these metal oxides with one another, and/or chemical mixtures (mixed oxides) of these metal oxides with aluminium oxide and/or chemical mixtures (mixed oxides) of these metal oxides with silicon dioxide. There is no restriction on the origin of the metal oxides. Thus metal oxides can be used which are derived from a pyrogenic process, in particular a flame hydrolysis process, a sol gel, a plasma process, a precipitation process, a hydrothermal process or by mining methods, or from combinations of the above processes.

Particularly preferred metal oxides are the pyrogenically produced metal oxides titanium dioxide, zinc oxide, iron oxide, cerium oxide, zirconium oxide and/or chemical mixtures (mixed oxides) of these metal oxides with one another, and/or chemical mixtures (mixed oxides) of these metal oxides with aluminium oxide and/or chemical mixtures (mixed oxides) of these metal oxides with silicon dioxide, at least one metal oxide being of pyrogenic origin.

The advantage of the process used is that there is no need for an organic solvent. In contrast to the process known according to EP-A-0 988 853, in which an organic solvent is absolutely essential to form the shell, in the process used according to the invention particles with a complete shell are obtained in a fast reaction.

The particles thus obtained are uniform, in other words only the particles used according to the invention are detected. Particles consisting exclusively of silicon dioxide, formed by the intergrowth of the fine SiO₂ particles formed during hydrolysis of the silicon dioxide source, cannot be detected. The metal oxides used according to the invention obviously have a high affinity to the silicon dioxide source.

The particles used according to the invention display a low structure and are therefore easy to incorporate into cosmetic formulations. These formulations are resistant to sedimentation.

The invention also provides sunscreens which contain the surface-modified oxide particles used according to the invention in a proportion of from 0.01 to 25 wt. %. The sunscreen according to the invention can also be used in blends with known inorganic UV-absorbing pigments and/or chemical UV filters.

Suitable examples of known UV-absorbing pigments are titanium dioxides, zinc oxides, aluminium oxides, iron oxides, silicon dioxide, silicates, cerium oxides, zirconium oxides, barium sulfate or mixtures thereof.

Suitable examples of chemical UV filters are all water- or oil-soluble UVA and UVB filters known to the person skilled in the art, for example sulfonic acid derivatives of benzophenones and benzimidazoles, derivatives of dibenzoyl methane, benzylidene camphor and derivatives thereof, derivatives of cinnamic acid and esters thereof, or esters of salicylic acid.

The sunscreens according to the invention can contain known solvents, such as water, monohydric or polyhydric alcohols; cosmetic oils; emulsifiers; stabilisers; consistency regulators, such as carbomers; cellulose derivatives; xanthan gum; waxes; bentones; pyrogenic silicas and other substances conventionally found in cosmetics, such as vitamins, antioxidants, preservatives, dyes and perfumes.

The sunscreen according to the invention can take the form of an emulsion (O/W, W/O or multiple), aqueous or aqueous-alcoholic gel or oil gel, and be produced in the form of lotions, creams, milk sprays, mousse, as a stick or in other common forms.

The procedure used for the production of sunscreen agents can be as described in A. Domsch, “Die kosmetischen Präparate”, Verlag für chemische Industrie (Ed. H. Ziolkowsky), 4^{th} edition, 1992 or N. J. Lowe and N. A. Shaat, Sunscreens, Development, Evaluation and Regulatory Aspects, Marcel Dekker Inc., 1990.

The invention also provides the use of the oxide particles according to the invention as UV filters, for the production of dispersions and use for chemical-mechanical polishing (CMP process).

### EXAMPLES

Examples 1-6 illustrate the production of the educts. Comparative examples 1-3 are performed in the presence of an organic solvent, ethanol. All examples include drying of the product after filtration at room temperature. A 29 wt. % aqueous ammonia solution is used as base.

The analytical data is contained in the table following the examples.

The composition of the core and shell is obtained by quantitative X-ray fluorescence analysis, the film thickness of the shell from the TEM images. The BET surface area is determined in accordance with DIN 66131 and the pore volume of the particles in accordance with DIN 66134. The hydroxyl group density is determined by the method disclosed by J. Mathias and G. Wannemacher in Journal of Colloid and Interface Science 125 (1998).

The dibutyl phthalate absorption is measured with a RHEOCORD 90 device supplied by Haake, Karlsruhe. To this end 16 g of the metal oxides described are introduced into a mixing chamber with an accuracy of 0.001 g, the chamber is closed with a lid and dibutyl phthalate is metered in through a hole in the lid at a predefined feed rate of 0.0667 ml/s. The compounder is operated at a motor speed of 125 revolutions per minute. On reaching the maximum torque, the compounder and DBP metering are automatically switched off. The DBP absorption is calculated from the amount of DBP consumed and the weighed amount of particles according to the formula below:

DBP value (ml/100 g)=(DBP consumption in ml/weighed amount of particles in g)×100.

FIG. 1A shows the typical behaviour of known pyrogenically produced oxides when a specific amount of dibutyl phthalate is added, with a sharply defined maximum and a subsequent drop. FIG. 1B shows the behaviour of the particles according to the invention. In this case a rise in torque with a subsequent drop when a specific amount of DBP is added cannot be seen. The dibutyl phthalate instrument detects no end point.

FIG. 2A shows a TEM image of the particles according to the invention produced in accordance with example 1; FIG. 2B shows a TEM image at the same magnification of the particles produced in accordance with comparative example 1. FIG. 2A shows the markedly lower degree of intergrowth of the particles according to the invention.

To determine the photocatalytic activity the sample to be measured is suspended in 2-propanol and irradiated with UV light for 1 hour. The concentration of acetone formed is then measured.

Approx. 250 mg (accuracy 0.1 mg) of the particles obtained from the examples and the comparative examples are suspended with an Ultra-Turrax stirrer in 350 ml (275.1 g) of 2-propanol. This suspension is pumped through a cooler maintained at a temperature of 24° C. into a glass photoreactor which has first been rinsed with oxygen and which has a radiation source.

An Hg medium-density immersion lamp, model TQ718 (Heraeus), for example, with a power of 500 W, is used as the radiation source. A protective tube made from borosilicate glass limits the emitted radiation to wavelengths>300 nm. The outside of the radiation source is surrounded by a cooling tube through which water is circulated.

Oxygen is metered into the reactor through a flow meter. When the radiation source is switched on, the reaction is started. At the end of the reaction a small amount of suspension is immediately removed, filtered and analysed by gas chromatography.

The speed constant for the formation of acetone, which follows a zero order kinetics in accordance with the equation dc(Ac)/dt=K, is stated.

### Example 1

100 g of titanium dioxide (P25 from Degussa) produced pyrogenically by flame hydrolysis are dispersed in 1 l of water. 100 ml of tetraethoxysilane are added to this solution. This mixture is stirred for 15 minutes, then 30 ml of ammonia are added. After stirring for 2-4 hours at 25° C. the product is filtered off and dried.

### Example 2

100 g of titanium dioxide (P25 from Degussa) produced pyrogenically by flame hydrolysis are dispersed in 1 l of water. 200 ml of tetraethoxysilane are added to this solution. This mixture is stirred for 15 minutes, then 30 ml of ammonia are added. After stirring for 2-4 hours at 25° C. the product is filtered off and dried.

### Example 3

100 g of titanium dioxide (P25 from Degussa) produced pyrogenically by flame hydrolysis are dispersed in 1 l of water. 100 ml of tetramethoxysilane are added to this solution. This mixture is stirred for 15 minutes, then 30 ml of ammonia are added. After stirring for 2-4 hours at 25° C. the product is filtered off and dried.

### Example 4

100 g of titanium dioxide (P25 from Degussa) produced pyrogenically by flame hydrolysis are dispersed in 1 l of water. 1000 ml of tetraethoxysilane are added to this solution. This mixture is stirred for 15 minutes, then 30 ml of ammonia are added. After stirring for 2-4 hours at 25° C. the product is filtered off and dried.

### Example 5

100 g of a titanium dioxide produced pyrogenically by flame hydrolysis, having a BET surface area of 100 m²/g, are dispersed in 1 l of water. 200 ml of tetraethoxysilane are added to this solution. This mixture is stirred for 15 minutes, then 30 ml of ammonia are added. After stirring for 2-4 hours at 25° C. the product is filtered off and dried.

### Example 6

100 g of titanium dioxide produced pyrogenically by flame hydrolysis and doped with 0.2% Al₂O₃ (produced as described in DE-A-196 50 500) are dispersed in 1 l of water. 200 ml of tetraethoxysilane are added to this solution. This mixture is stirred for 15 minutes, then 30 ml of ammonia are added. After stirring for 2-4 hours at 25° C. the product is filtered off and dried.

### Comparative Example 1

100 g of titanium dioxide (P25 from Degussa) produced pyrogenically by flame hydrolysis are dispersed in 1.5 l of ethanol and 100 ml of water. 50 ml of ammonia are added to this solution. 100 ml of tetraethoxysilane in 200 ml of ethanol are then slowly added dropwise to this mixture over a period of 1 hour. After 12 hours the product is filtered off and dried.

### Comparative Example 2

400 ml of water, 1388 ml of ethanol and 87 ml of ammonia are mixed together, then 105 g of titanium dioxide are dispersed therein. 193 ml of tetraethoxysilane in 24 ml of water and 156 ml of ethanol are added to this solution over a period of 6 hours. The dispersion is aged for a further 12 hours at 25° C. The product is filtered off and dried.

### Comparative Example 3

106 ml of water, 480 ml of ethanol and 20 ml of ammonia are mixed together, then 28 g of titanium dioxide are dispersed therein. 105 ml of tetraethoxysilane in 39.5 ml of water and 65.5 ml of ethanol are added to this solution over a period of 2 hours. The dispersion is aged for a further 12 hours at 20° C. The product is then recovered by filtration and dried.

The products according to examples 1 and 3 are subsequently used as educts for surface modification.

TABLE

| | | | | DBF | k | BET | OH | Pore |
|---|---|---|---|---|---|---|---|---|
| | Core¹ | Shell² | Shell² | absorption | [10⁻³ mol | surf. a. | density | volume |
| | [wt. %] | [wt. %] | [nm] | [ml/100 g] | kg⁻¹min⁻¹] | [m²/g] | [OH/nm²] | [cm³/g] |
| Example | | | | | | | | |
| P25³ | 99.5 | — | — | 96 | 0.68 | 50 | 23.2 | — |
| 1 | 88.1 | 11.7 | 2 | No end point | 0.08 | 64 | 8.1 | 0.12 |
| 2 | 80.0 | 20 | 3-4 | No end point | 0.10 | 80 | 4.4 | 0.16 |
| 3 | 85.7 | 14.3 | 3 | No end point | 0.14 | 75 | 5.2 | 0.14 |
| 4 | 37.7 | 62.3 | 16-18 | No end point | 0.09 | 63 | 4.9 | 0.15 |
| 5⁴ | 63.7 | 36.2 | 7 | No end point | 0.12 | 123 | 5.3 | 0.21 |
| 6⁵ | 80.1 | 18.7 | 3-4 | No end point | 0.16 | 77 | 6.2 | 0.15 |
| Comparative | | | | | | | | |
| example | | | | | | | | |
| 1 | 80.2 | 19.8 | 3 | ns | 0.28 | 40 | — | 0.08 |
| 2 | 68.1 | 31.9 | 10 | ns | 0.42 | 40 | 2.2 | 0.07 |
| 3 | ns⁶ | ns | 3 | ns | 0.38 | 35 | ns | 0.06 |

¹Core: Examples 1 to 4, all comparative examples: TiO₂ with approx. 50 m²/g BET;
²Shell: Examples 1, 2, 4-6, all comparative examples: SiO₂ source: Si(OEt)₄, example 3: Si(OMe)₄
³P25: Pyrogenic titanium dioxide, Degussa
⁴TiO₂ with approx. 100 m²/g
⁵TiO₂ doped with 0.1 wt. % Al₂O₃ with approx. 50 m²/g BET
⁶ns = not specified

### Production of the Products

The coated titanium oxides are placed in a mixer for surface modification and sprayed first with water (optionally) and then with the surface modifying agent whilst undergoing intensive mixing.

On completion of spraying, mixing can be continued for a further 15 to 30 minutes and the mixture can then be conditioned for 1 to 4 hours at 50 to 400° C.

The water used can be acidulated with an acid, for example hydrochloric acid, to obtain a pH of 7 to 1. The surface modifying agent used can be dissolved in a solvent, such as ethanol for example.

The products obtained display the data set out in Table 2.

TABLE 1
Surface modification of the coated titanium dioxides

| | Example according to the invention | |
|---|---|---|
| | 1 | 2 |
| Oxide | Example 1 | Example 3 |
| Silane | Octyl | Octyl |
| | trimethoxysilane | trimethoxysilane |
| | (Dynasylan OCTMO) | (Dynasylan OCTMO) |
| Parts silane/ | 6 | 6 |
| 100 parts oxide | | |
| Parts H₂O/ | 1 | 1 |
| 100 parts oxide | | |
| Conditioning | 120 | 120 |
| temperature [° C.] | | |
| Conditioning time | 2 | 2 |
| [h] | | |

TABLE 2
Physico-chemical data for the surface-modified
products from Table 1

Example according to the invention

| | Example 1 | Example 2 |
|---|---|---|
| BET surface area [m²/g] | 48 | 61 |
| C content [%] | 1.5 | 1.2 |
| Loss on drying [%] | 0.2 | 0.5 |
| Loss on ignition [%] | 2.7 | 1.5 |
| pH | 7.9 | 7.3 |

The surface-modified, coated titanium dioxides according to the invention display the following properties:

The photocatalytic activity of the titanium dioxides is largely eliminated by the surface modification. The photocatalytic activity is determined as described above (photochemical oxidation of isopropanol to acetone).

The K values are 0.04 (example 1 according to the invention) and 0.002 (example 2 according to the invention), in comparison to 0.08 to 0.16×10⁻³ mol/kg min for the non-surface-modified, coated titanium dioxides. The photocatalytic activity is thus reduced still further.

### Sunscreen

A sunscreen containing 4 wt. % of the particles according to the invention in accordance with example 2 is produced using the formulation below.

| Phase | Component | wt. % |
|---|---|---|
| A | Isolan GI 34 | 3.0 |
| | Castor oil | 1.2 |
| | Tegesoft OP | 10.0 |
| | Tegesoft Liquid | 5.0 |
| | Glycerol 86% | 3.0 |
| B | Paracera W80 | 1.8 |
| | Isohexadecane | 5.0 |
| C | Particles according to | 4.0 |
| | the invention in | |
| | accordance with example 2 | |
| D | Magnesium sulfate | 0.5 |
| | Demineralised water | 66.5 |

Phase A is heated in a mixer to 70° C. After melting on a magnetic hotplate at 80° C., phase B is added to phase A. Phase C is stirred into the oil phase at approx. 300 rpm under vacuum. Phase D is likewise heated to 70° C. and added to the mixture of A to C under vacuum.

Sunscreen creams are produced with the surface-modified, coated titanium dioxides in the same way as in the formulation above. These sunscreen creams are characterised by a good skin feel and low whitening.

The surface-modified, coated non-metal/metal oxides according to the invention advantageously display
very low photocatalytic activity (hence no degradation of the formulation when exposed to light, for example)
very good dispersability (hence good incorporation ability, high UV protection, good skin feel, low whitening when applied to the skin)
high water resistance (important for beach products).

1. Surface-modified, coated oxide particles consisting of a core of a metal oxide and a shell of silicon dioxide surrounding the core, wherein the coated oxide particles display a low structure, defined by the absence of an end point in dibutyl phthalate absorption.

2. Surface-modified oxide particles according to claim 1, characterised in that the BET surface area is between 5 and 600 m²/g.

3. Surface-modified oxide particles according to claim 1, characterised in that the primary particle size is between 2 and 100 nm and the secondary particle size is between 0.05 and 50 μm.

4. Surface-modified oxide particles according to claim 1, characterised in that the film thickness of the silicon dioxide shell is between 0.5 and 25 nm.

5. Surface-modified oxide particles according to claim 1, characterised in that the metal oxides are selected from the group consisting of titanium dioxide, zinc oxide, zirconium oxide, iron oxide, cerium oxide, chemical mixtures (mixed oxides) of these metal oxides with one another, chemical mixtures (mixed oxides) of these metal oxides with aluminium oxide and chemical mixtures (mixed oxides) of these metal oxides with silicon dioxide.

6. Surface-modified oxide particles according to claim 1, characterised in that the metal oxides are selected from the group consisting of pyrogenic titanium dioxide, pyrogenic zinc oxide, pyrogenic zirconium oxide, pyrogenic iron oxide, pyrogenic cerium oxide, chemical mixtures (mixed oxides) of these metal oxides with one another, chemical mixtures (mixed oxides) of these metal oxides with aluminium oxide, chemical mixtures (mixed oxides) of these metal oxides with silicon dioxide, wherein in mixtures at least one metal oxide is of pyrogenic origin.

7. A process for producing the surface-modified oxide particles according to claim 1, comprising adding a base dissolved in water with stirring to a dispersion of 1-80 wt. % of a metal oxide, at least one compound of the type XₙSi(OR)₄₋ₙ, wherein the molar ratio XₙSi(OR)₄₋ₙ/metal oxide is between 0.1 and 25, depending on the film thickness of the silicon dioxide shell, and water, separating the reaction product off, optionally washing, drying and then surface modifying.

8. The process according to claim 7, wherein the metal oxides are selected from the group consisting of titanium dioxide, zinc oxide, zirconium oxide, iron oxide, cerium oxide, chemical mixtures (mixed oxides) of these metal oxides with one another, chemical mixtures (mixed oxides) of these metal oxides with aluminium oxide, and chemical mixtures (mixed oxides) of these metal oxides with silicon dioxide.

9. The process according to claim 7, wherein the metal oxides are selected from the group consisting of pyrogenic titanium dioxide, pyrogenic zinc oxide, pyrogenic zirconium oxide, pyrogenic iron oxide, pyrogenic cerium oxide, chemical mixtures (mixed oxides) of these metal oxides with one another, chemical mixtures (mixed oxides) of these metal oxides with aluminium oxide, and chemical mixtures (mixed oxides) of these metal oxides with silicon dioxide, wherein in mixtures at least one metal oxide is of pyrogenic origin.

10. Process according to claim 7, wherein compounds of the type XₙSi(OR)₄₋ₙ can be those in which X=halogen, R=H or a linear or a branched alkyl radical having 1 to 8 C atoms and n=0 to 4, where R does not equal H if n=4.

11. Sunscreen containing the oxide particles according to claim 1 in a proportion of between 0.01 and 25 wt. %, relative to the amount of sunscreen.

12. A dispersion comprising water and the oxide particles according to claim 1.

13. Surface-modified oxide particles according to claim 2, characterised in that the primary particle size is between 2 and 100 nm and the secondary particle size is between 0.05 and 50 μm.

14. Surface-modified oxide particles according to claim 2, characterised in that the film thickness of the silicon dioxide shell is between 0.5 and 25 nm.

15. Surface-modified oxide particles according to claim 3, characterised in that the film thickness of the silicon dioxide shell is between 0.5 and 25 nm.

16. Surface-modified oxide particles according to claim 2, characterised in that the metal oxides are selected from the group consisting of titanium dioxide, zinc oxide, zirconium oxide, iron oxide, cerium oxide, chemical mixtures (mixed oxides) of these metal oxides with one another, chemical mixtures (mixed oxides) of these metal oxides with aluminium oxide and chemical mixtures (mixed oxides) of these metal oxides with silicon dioxide.

17. Surface-modified oxide particles according to claim 2, characterised in that the metal oxides are selected from the group consisting of pyrogenic titanium dioxide, pyrogenic zinc oxide, pyrogenic zirconium oxide, pyrogenic iron oxide, pyrogenic cerium oxide, chemical mixtures (mixed oxides) of these metal oxides with one another, chemical mixtures (mixed oxides) of these metal oxides with aluminium oxide, chemical mixtures (mixed oxides) of these metal oxides with silicon dioxide, wherein in mixtures at least one metal oxide is of pyrogenic origin.
